(19) United States
(12) Patent Application Publication
(10) Pub. No.: US 2021/0386956 A1
(43) Pub. Date: Dec. 16, 2021

(54) FILTER WITH COMBINED WEAR INDICATION AND PULL TAB

(71) Applicant: KONINKLIJKE PHILIPS N.V., Eindhoven (NL)

(72) Inventors: MARK WILLIAM DIMATTEO, IRWIN, PA (US); MARK WAYNE BARCLAY, PITTSBURGH, PA (US); MATTHEW SCOTT ANSWINE, APOLLO, PA (US)

(21) Appl. No.: 17/126,082

(22) Filed: Dec. 18, 2020

Related U.S. Application Data
(60) Provisional application No. 62/953,244, filed on Dec. 24, 2019.

(51) Int. Cl. A61M 16/10; A61M 16/00; A61M 16/06

(52) U.S. Cl. CPC A61M 16/107; A61M 2016/003; A61M 16/06; A61M 16/0066

(57) ABSTRACT

A filter assembly for filtering a flow of incoming air entering a pressurized breathing gas system includes a filter housing and a filter media. The filter media includes a filtering section and a non-filtering section. The filter housing is structured to be inserted within a pressure generating device used in the pressurized breathing gas system. The filtering section is disposed within the filter housing and is structured to filter contaminant matter from the flow of incoming air. The non-filtering section is disposed outside of the housing and is structured to be separated from the filtering section so as to not make contact with the flow of incoming air. The filtering section and the non-filtering section are structured to be visually compared to one another such that a contaminant matter saturation level of the filter media can be determined.

### 1. FIELD OF THE INVENTION

The present invention pertains to pressurized breathing gas systems, and, more particularly, to filters used in pressurized breathing gas systems, and a method for determining when replacement of said filters is necessary.

### 2. DESCRIPTION OF THE RELATED ART

Many individuals suffer from disordered breathing during sleep. Sleep apnea is a common example of such sleep disordered breathing suffered by millions of people throughout the world. It is known to deliver positive airway pressure (PAP) to treat a medical disorder, such as chronic obstructive pulmonary disease (COPD) or sleep apnea syndrome, in particular, obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle.

Pressurized breathing gas therapies such as CPAP involve the placement of a patient interface device including a mask component on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. The patient interface device interfaces the ventilator or pressure support device with the airway of the patient, so that a flow of breathing gas can be delivered from a pressure/flow generating device to the airway of the patient. It is known to maintain such devices on the face of a wearer by a headgear having one or more straps adapted to fit over/around the patient's head.

Air filters, specifically air inlet filters, are an important part of airway pressure support systems. Not only do they protect the inner workings of the device by preventing foreign matter from entering the unit, but they also protect the patient from airborne contaminants. In the current airway pressure support system market, air filters are typically die cut pieces of filter media that sit at the air inlet of the device.

There are two types of air filters that are commonly used in airway pressure support systems. The first type of filter, referred to as a coarse particle filter, is structured to trap and filter relatively large pieces of gross particulate matter from the air before it enters the airway pressure support system. The second type of filter, referred to as a fine particle filter, is designed to be employed in combination with a coarse particle filter and is structured to trap and filter smaller pieces of particulate matter and airborne contaminants that would not otherwise be filtered by the coarse particle filter. Use of a fine particle filter in an airway pressure support system is typically optional. Thus, in practice, an airway pressure support system may be used with a coarse particle filter alone or with a combination of a coarse particle filter and a fine particle filter. When used in combination, the course particle filter and fine particle filter are placed in series with one another.

Visually determining when a filter is saturated with particulate matter and needs to be replaced can be difficult. This is especially true if the filter is dark in color and the saturated media is not much different in contrast than the original, unused media. Even when the filter media is light in color, e.g. white, there may not be an obvious change in color of the media to indicate that it is time to change the filter.

There is thus a need for a mechanism for use with pressurized breathing gas systems which makes it readily apparent when a filter media needs to be changed.

### SUMMARY OF THE INVENTION

Accordingly, one or more embodiments provide a filter assembly configured to facilitate ease in visually determining when a filter media for a pressurized breathing gas system needs to be changed, by using one section of the filter media to filter a flow of incoming air to the system and isolating another non-filtering section of the media from the flow of incoming air, such that a difference in the contaminant saturation level of the filtering section compared to the non-filtering section is apparent. In one embodiment, a filter assembly for filtering incoming air entering a pressurized breathing gas system includes a filter housing and a filter media. The filter media includes a filtering section and a non-filtering section. The filter housing is structured to be inserted within a pressure generating device used to generate a flow of pressurized air for delivery to an airway of a user of the pressurized breathing gas system. The filtering section is disposed within the filter housing. The non-filtering section is disposed outside of the filter housing. The filter housing comprises a filtering boundary structured to separate the non-filtering section from the filtering section such that the non-filtering section is isolated from the flow of incoming air. The filtering section and the non-filtering section are structured to be visually compared to one another such that a contaminant matter saturation level of the filter media can be determined.

In another embodiment, a method for filtering incoming air entering a pressurized breathing gas system includes: providing a filter assembly including a filter housing and a filter media, the filter media including a filtering section and a non-filtering section; disposing the filtering section within the filter housing and disposing the non-filtering section outside of the filter housing; inserting the filter assembly within a pressure generating device used with the pressurized breathing gas system; filtering contaminant matter from the flow of incoming air with the filtering section; separating the non-filtering section from the filtering section such that the non-filtering section is isolated from the flow of incoming air; and determining a contaminant matter saturation level of the filter media by visually comparing the filtering section to the non-filtering section.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 is a is a schematic diagram of a pressurized breathing gas system which employs the use of a filter assembly according to an exemplary embodiment of the disclosed concept;

FIG. 2 is an illustration depicting how a filter assembly is inserted into a pressure generating device for a pressurized breathing gas system according to an exemplary embodiment of the disclosed concept;

FIGS. 3A-G show plan, side, and perspective views of a filter assembly according to an exemplary embodiment of the disclosed concept; and

FIGS. 4A-4E are illustrations of a filter assembly according to an exemplary embodiment of the disclosed concept.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

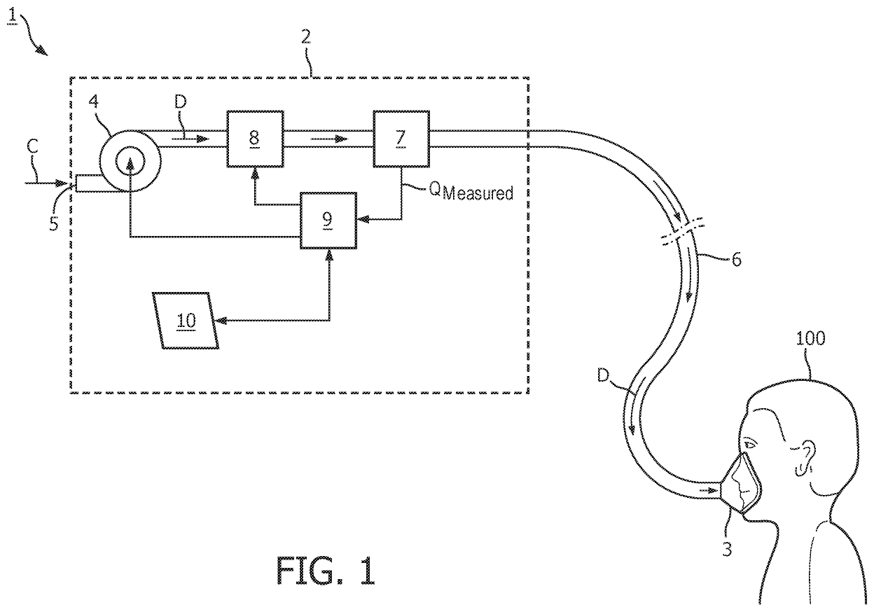

FIG. 1 shows a schematic diagram of pressurized breathing gas system 1. Pressurized breathing gas system 1 includes a pressure generating device 2 for delivering a flow of breathing gas to a patient 100 through a mask 3, which is typically worn by or otherwise attached to patient 100 to communicate the flow of breathing gas to the airway of patient 100. In the illustrated exemplary embodiment shown in FIG. 1, mask 3 is a nasal/oral mask. It will be appreciated, however, that mask 3 can be a nasal mask, a pillows style nasal cushion, a cradle style nasal cushion, a full face mask, or any other patient interface device that provides a suitable gas flow communicating function without departing from the scope of the present invention.

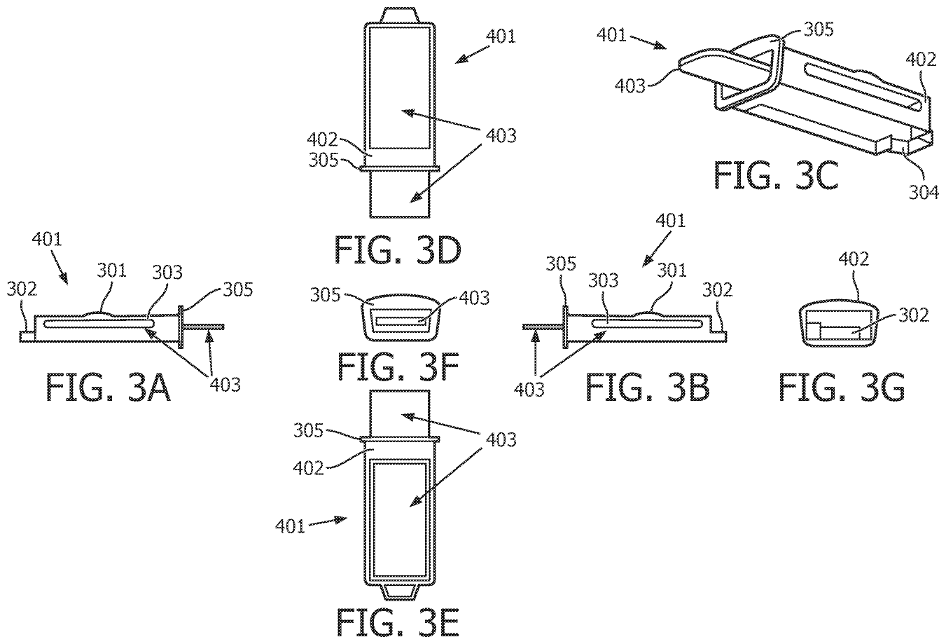

Pressure generating device 2 includes a gas flow generator 4, such as a blower used in a conventional CPAP or bi-level pressure support device, which receives breathing gas, generally indicated by arrow C, from any suitable source, e.g., a pressurized tank of oxygen or air, the ambient atmosphere, or a combination thereof. Gas flow generator 4 generates a flow of breathing gas, such as air, oxygen, or a mixture thereof, for delivery to an airway of patient 100 at relatively higher and lower pressures, i.e., generally equal to or above ambient atmospheric pressure. A filter assembly 401 (shown in FIGS. 3A-3E) is disposed within gas flow generator 4 near opening 5 of gas flow generator 4 such that breathing gas C can be filtered before delivery to the airway of patient 100. The pressurized flow of breathing gas, generally indicated by arrow D from gas flow generator 4, is delivered via a delivery conduit 6 to mask 3.

Pressurized breathing gas system 1 further includes flow sensor 7 that measures the flow of the breathing gas within delivery conduit 6. In the particular embodiment shown in FIG. 1, flow sensor 7 is interposed in line with delivery conduit 6, most preferably downstream of valve 8, which controls pressure. Flow sensor 7 generates a flow signal Q_{MEASURED} that is provided to controller 9 and is used by controller 9 to determine the rate of flow of gas at patient 100, referred to as Q_{PATIENT}. It will be appreciated that a pressure generating device 2 may employ other configurations of pressure control and flow sensing without departing from the scope of the disclosed concept.

Controller 9 includes a processing unit, such as, for example, a microprocessor, a microcontroller or some other suitable processing device, and a memory (that is provided as part of the processing unit or that is operatively coupled to the processing unit) that provides a tangible storage medium for data and software routines executable by the processing unit for controlling the operation of pressurized breathing gas system 1. Input/output unit 10 is provided for setting various parameters used by pressurized breathing gas system 1, as well as for displaying and outputting information and data to a user, such as a clinician or caregiver. It will be appreciated that input/output unit 9 may include physical buttons, turn knobs, or any other means for enabling a user to enter input into input/output unit 10 without departing from the scope of the disclosed concept.

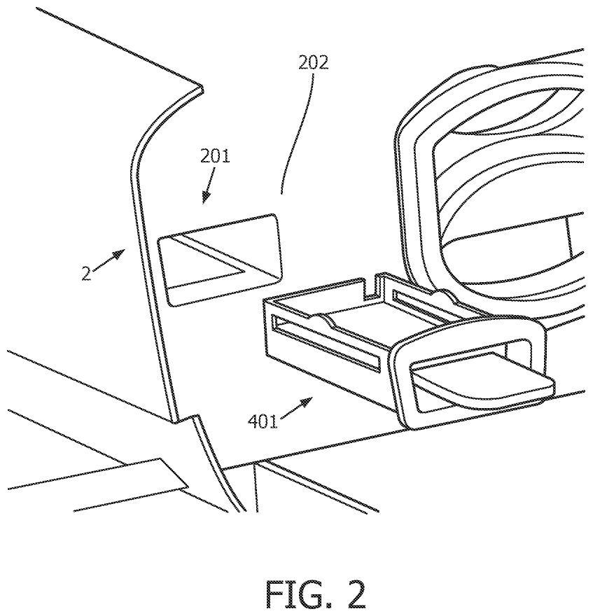

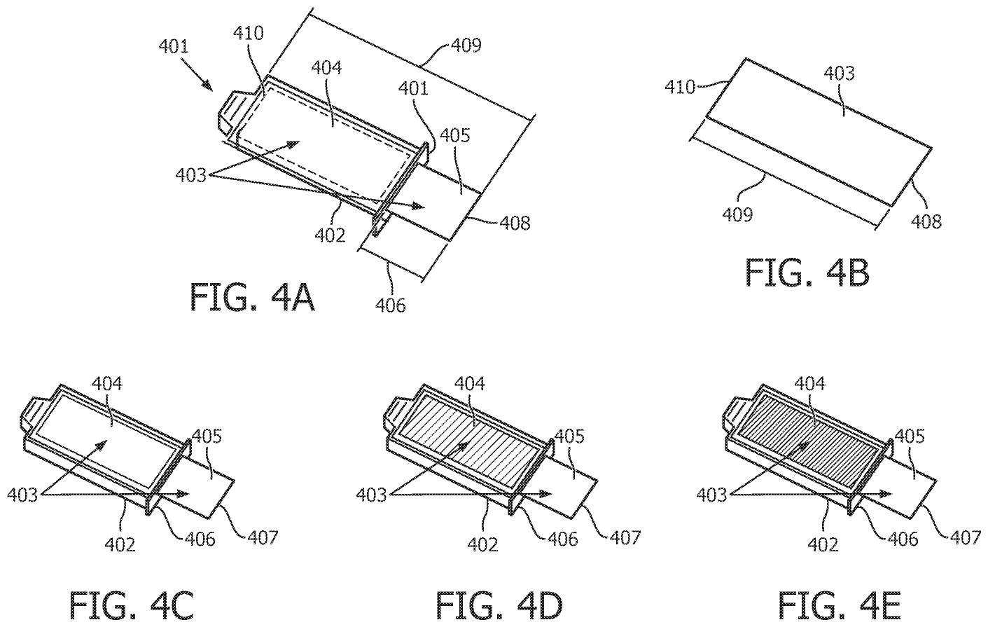

FIG. 2 is an illustration depicting how a filter assembly 401 is inserted into a pressure generating device 2 according to an exemplary embodiment of the disclosed concept. Filter assembly is also shown in FIGS. 4A-4E and includes filter housing 402 and filter media 403. Filter housing 402 of filter assembly 401 is structured to engage with slot 201 of pressure generating device 2 such that filter assembly 401 fits securely within slot 201. A secure fit of filter housing 402 within slot 201 is facilitated by including features (shown in FIGS. 3A-3F) on filter housing 402 that are complementary to slot 201 and vice versa.

FIG. 3A shows a left side view of filter assembly 401 and FIG. 3B shows a right side view of filter assembly 401. In one exemplary embodiment, protrusions 301 may be formed on filter housing 402 as a feature to facilitate the engagement of a secure fit of filter assembly 401 within slot 201. In this exemplary embodiment, slot 201 may be formed with depressions such that protrusions 301 would mate with the depressions when filter assembly 401 is inserted completely into slot 201. In another exemplary embodiment, a shelf 302 may be formed on a back side of filter housing 402 as a feature to facilitate the engagement of a secure fit of filter housing 402 within slot 201. In this exemplary embodiment, slot 201 may be formed with a notch such that shelf 302 would mate with the notch when filter assembly 401 is inserted completely into slot 201. In another exemplary embodiment, channels 303 may be formed on the sides of filter housing 402 as a feature to facilitate the engagement of a secure fit of filter assembly 401 within slot 201. In this exemplary embodiment, slot 201 may be formed with projections such that channels 303 would mate with the projections when filter assembly 401 is inserted completely into slot 201.

FIG. 3C shows a perspective view of filter assembly 401. In another exemplary embodiment, filter housing 402 may be formed with boundary extension 304 such that boundary extension 304 extends below the plane of filter 403. In this exemplary embodiment, slot 201 may be formed with a boundary groove such that boundary extension 304 would mate with the boundary groove when filter assembly 401 is inserted completely into slot 201. In another exemplary embodiment, enlargement 305 is formed on filter housing 402. In this exemplary embodiment, pressure generating device 2 would be formed with a depressed ring 202 around slot 201 such that enlargement 305 would mate with depressed ring 202 when filter assembly 401 is inserted completely into slot 201. FIG. 3D shows a plan view of the top side of filter assembly 401, FIG. 3E shows a plan view of the bottom side of filter assembly 401, FIG. 3F shows a front side view of filter assembly 401, and FIG. 3G shows a back side view of filter assembly 401. While FIG. 2 and FIGS. 3A-G illustrate protrusions 301, shelf 302, channels 303, extension 304, enlargement 305, and depressed ring 202 as features for facilitating a secure fit of filter assembly 401 within slot 201, it will be appreciated that other features may be used to facilitate a secure fit of filter assembly 401 within slot 201 without departing from the scope of the disclosed concept.

FIG. 4A is an illustration of a filter assembly 401 according to an exemplary embodiment of the disclosed concept. Filter assembly 401 includes filter housing 402 and filter media 403. Filter media 403 is flexible, while also comprising a geometric plane. The dimensions and disposition of filter media 403 within filter housing 402 are such that a filtering section 404 of filter media 403 is disposed within filter housing 402 and a non-filtering section 405 of filter media 403 is disposed outside of filter housing 402. Filtering section 404 performs the function of filtering the flow of incoming air in pressure generating device 2 (breathing gas C shown in FIG. 1), while non-filtering section 405 is isolated from the flow of incoming air and does not perform the filtering function. Because non-filtering portion 405 of filter media 403 is isolated from the flow of incoming air, when filter assembly 401 is removed from slot 201, the contaminant saturation level of filtering section 404 is readily apparent when filtering section 404 is visually compared to non-filtering section 405. In addition to serving as a reference for the contaminant saturation level of filtering section 404, non-filtering portion 405 serves the purpose of acting as a pull tab that facilitates ease of removal of filter media 403 from filter housing 402. In one exemplary embodiment of the disclosed concept, a length 406 of non-filtering portion 405 measures at least 1 cm long from an edge 407 of filter housing 402 to an edge 408 of non-filtering portion 405. In an alternative exemplary embodiment of the disclosed concept, the length 406 of non-filtering portion 405 is at least 20% of the overall length 409 of filter media 403 from edge 408 to an opposite edge 410. However, it will be appreciated that length 406 may be of any length that permits a user to effectively use non-filtering portion 405 as a pull tab for removing filter media 403 from filter housing 402 without departing from the scope of the disclosed concept.

FIG. 4C illustrates an unused filter and shows no contrast between filtering section 404 and non-filtering section 405. FIG. 4D illustrates a mid-life filter and shows moderate contrast between filtering section 404 and non-filtering section 405. FIG. 4E illustrates an end-life filter and shows maximum contrast between filtering section 404 and non-filtering section 405. In an exemplary embodiment, filtering section 404 and non-filtering section 405 are separated by a seal to isolate non-filtering section 405 from the flow of incoming air, and said seal could be formed by over-molding filter housing 402 around filter media 403. It will be appreciated, however, that filter section 404 and non-filtering section 405 may be separated by other means to isolate non-filtering section 405 from the flow of incoming air without departing from the scope of the disclosed concept.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although this description includes details for the purpose of illustration based on what is currently considered to be the most practical and preferred embodiments, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that, to the extent possible, one or more features of any embodiment are contemplated to be combined with one or more features of any other embodiment.

What is claimed is:

1. A filter assembly for filtering a flow of incoming air entering a pressurized breathing gas system, comprising:
a filter housing; and
a filter media, comprising:
a filtering section; and
a non-filtering section,
wherein the filtering section is disposed within the filter housing,
wherein the non-filtering section is disposed outside of the filter housing,
wherein the filter housing is structured to be inserted within a pressure generating device used to generate a flow of pressurized air for delivery to an airway of a user of the pressurized breathing gas system,
wherein the filtering section is structured to filter contaminant matter from the flow of incoming air,
wherein the filter housing comprises a filtering boundary structured to separate the non-filtering section from the filtering section such that the non-filtering section is isolated from the flow of incoming air, and
wherein the filtering section and the non-filtering section are structured to be visually compared to one another such that a contaminant matter saturation level of the filter media can be determined.

2. The filter assembly of claim 1, wherein the filtering boundary comprises a seal.

3. The filter assembly of claim 2, wherein the seal is formed by over-molding the housing around the filtering section.

4. The filter assembly of claim 1, wherein the non-filtering section is in a same geometric plane as the filtering section.

5. The filter assembly of claim 1, wherein the non-filtering section is structured to be used as a pull tab such that the filter media can be removed from the filter housing by pulling the non-filtering section away from the filter housing.

6. The filter assembly of claim 5,
wherein a first edge of the non-filtering section is formed by the filtering boundary,
wherein a second edge of the non-filtering section comprises an edge of the non-filtering section disposed opposite of the first edge, and
wherein a distance from the first edge of the non-filtering section to the second edge of the non-filtering section measures at least 1.0 centimeter.

7. The filter assembly of claim 5,
wherein a first edge of the non-filtering section is formed by the filtering boundary,
wherein a second edge of the non-filtering section comprises an edge of the non-filtering section disposed opposite of the first edge of the non-filtering section,
wherein the second edge of the non-filtering section comprises an outer edge of the filter media,
wherein an inner edge of the filter media, disposed within the filter housing, comprises an edge of the filter media opposite the outer edge of the filter media,
wherein a length of the non-filtering section comprises a distance measured from the first edge of the non-filtering section to the second edge of the non-filtering section,
wherein a length of the filter media comprises a distance measured from the inner edge of the filter media to the outer edge of the filter media, and
wherein the length of the non-filtering section measures at least 20% of the length of the filter media.

8. A method for filtering a flow of incoming air entering a pressurized breathing gas system, comprising:
providing a filter assembly, comprising:
a filter housing; and
a filter media, comprising:
a filtering section; and
a non-filtering section;
disposing the filtering section within the filter housing;
disposing the non-filtering section outside of the filter housing;
inserting the filter assembly within a pressure generating device used to generate a flow of pressurized air for delivery to an airway of a user of the pressurized breathing gas system,
filtering contaminant matter from the flow of incoming air with the filtering section;
separating the non-filtering section from the filtering section with a filtering boundary of the filter housing such that the non-filtering section is isolated from the flow of incoming air; and
determining a contaminant matter saturation level of the filter media by visually comparing the filtering section to the non-filtering section.

9. The method of claim 8, wherein the filtering boundary comprises a seal.

10. The method of claim 9, wherein the seal is formed by over-molding the housing around the filtering section.

11. The method of claim 8, wherein the non-filtering section is in a same geometric plane as the filtering section.

12. The method of claim 8, further comprising:
structuring the non-filtering section to be used as a pull tab such that the filter media can be removed from the filter housing by pulling the non-filtering section away from the filter housing.

13. The method of claim 12,
wherein a first edge of the non-filtering section is formed by the filtering boundary,
wherein a second edge of the non-filtering section comprises an edge of the non-filtering section disposed opposite of the first edge, and
wherein a distance from the first edge of the non-filtering section to the second edge of the non-filtering section measures at least 1.0 centimeter.

14. The method of claim 12,
wherein a first edge of the non-filtering section is formed by the filtering boundary,
wherein a second edge of the non-filtering section comprises an edge of the non-filtering section disposed opposite of the first edge of the non-filtering section,
wherein the second edge of the non-filtering section also comprises an outer edge of the filter media,
wherein an inner edge of the filter media, disposed within the filter housing, comprises an edge of the filter media opposite the outer edge of the filter media,
wherein a length of the non-filtering section comprises a distance measured from the first edge of the non-filtering section to the second edge of the non-filtering section,
wherein a length of the filter media comprises a distance measured from the inner edge of the filter media to the outer edge of the filter media, and
wherein the length of the non-filtering section measures at least 20% of the length of the filter media.
